(19) United States
(12) Patent Application Publication
(10) Pub. No.: US 2022/0023220 A1
(43) Pub. Date: Jan. 27, 2022

(54) RAPIDLY DISINTEGRATING ORAL TABLET

(71) Applicant: Columbia Care LLC, New York, NY (US)

(72) Inventors: Aaron M. Dely, Lighthouse Point, FL (US); Shaun Ben-Ari, Rochester, NY (US)

(21) Appl. No.: 17/421,916

(22) PCT Filed: Jan. 10, 2020
(86) PCT No.: PCT/US2020/013120

§ 371 Date: Jul. 9, 2021

Related U.S. Application Data
(60) Provisional application No. 62/790,831, filed on Jan. 10, 2019.

(51) Int. Cl. A61K 9/20; A61K 36/185; A61K 31/05; A61K 31/352; A61K 9/00

(52) U.S. Cl. CPC A61K 9/2054; A61K 36/185; A61K 31/05; A61K 9/0056; A61K 9/2018; A61K 9/2009; A61K 9/2059; A61K 31/352

(57) ABSTRACT

Disclosed are cannabinoid formulations and methods of use associated therewith. Specifically, disclosed are rapidly disintegrating or dissolving tablets for oral administration comprising one or more cannabinoids or cannabinoid extracts.

### RELATED APPLICATIONS

This application claims the benefit of priority to U.S. Provisional Application No. 62/790,831, filed Jan. 10, 2019, the contents of which are incorporated herein by reference in their entirety.

### FIELD OF INVENTION

Disclosed are cannabinoid formulations and methods of use associated therewith. Specifically, disclosed are rapidly disintegrating or dissolving tablets for oral administration containing one or more cannabinoids or cannabinoid extracts.

### BACKGROUND OF THE INVENTION

The medicinal and psychoactive properties of the cannabis plant have been known for centuries. While it has been illegal in many countries, there is a growing population lobbying for legalization of its use, especially for medicinal purposes.

Cannabis is believed to provide benefits in the treatment of multiple disorders with safer and fewer serious side effects than most prescription drugs currently used as antiemetics, muscle relaxants, hypnotics, and analgesics. A disadvantage in treating patients with cannabis is the psychoactive effect, especially in “naive” cannabis users. Furthermore, there have been reports of unpleasant reactions to cannabis, such as anxiety, panic, or hallucinations. It is believed that the undesirable side effects are most commonly associated with higher doses of cannabis and are related to the difficulty in controlling the dosage when the drug is smoked or eaten in cannabis-enriched confectionaries.

Cannabis has also been used to treat the symptoms in patients suffering from serious medical conditions. For example, cannabis has been used to alleviate symptoms associated with cancer, anorexia, AIDS, chronic pain, muscle spasticity, glaucoma, arthritis, migraine, and many other illnesses. Cannabis is recognized as having antiemetic properties and has been successfully used to treat nausea and vomiting in cancer patients undergoing chemotherapy. Cannabis has also been used in treating the weight loss syndrome of AIDS and in treating glaucoma by reducing intraocular pressure. Cannabis is also known for its muscle relaxing and anti-convulsant effects.

The most prevalent mode of administration of medical cannabis is by smoking. This mode of administration can have adverse effects on the lungs. Cannabis smoke carries more tar and other particulate matter than tobacco and may be a cause of lung diseases including lung cancer. Furthermore, many patients find the act of smoking unappealing, as well as generally unhealthy.

Accordingly, there is significant interest in developing other means to administer cannabis to patients.

For design of an orally disintegrating tablet, a composition desirably maintains enough porosity inside the compressed tablets for fast dissolving or fast melting while maintaining the mechanical strength of the tablet.

Current technologies involved in many patents as well as existing commercial fast-dissolving tablets utilize complicated processing techniques such as freeze-drying, molding, and sublimation or use of specialized excipients such as effervescent couple, highly micronized agents or the like.

There remains an unmet need for a rapidly dissolving tablet containing cannabinoids for oral administration.

### SUMMARY OF THE INVENTION

The present application relates to the selective use of excipients that maintain a fast rate of dissolution of the cannabinoids.

In one aspect, provided are formulations comprising at least one cannabinoid or a derivative thereof, a solvent, and a pharmaceutically acceptable adsorbent.

In another aspect, provided are methods for treating a disease, or a disorder, the methods comprising administering, e.g., to a subject, a formulation, e.g., a pharmaceutical formulation, disclosed herein.

In yet another aspect, provided are methods of manufacturing a formulation comprising the steps of: providing one or more cannabinoids or derivatives thereof, providing one or more solvents, providing one or more pharmaceutically acceptable adsorbents, providing one or more fillers, excipients, disintegrants, or combinations thereof, and mixing, e.g., mixing the one or more cannabinoids or derivatives thereof, the one or more solvents, the one or more pharmaceutically acceptable adsorbents, and the one or more fillers, excipients, disintegrants, or combinations thereof.

In one aspect, provided herein are formulations comprising:

a. at least one cannabinoid or a derivative thereof,

b. a solvent, and

c. a pharmaceutically acceptable adsorbent.

Exemplary solvents include a vegetable oil, hydrogenated vegetable oils, coconut oil, limonene, a terpene, an essential oil, polyethylene glycols (PEG), propylene glycol, ethanol, substituted polyethylene glycols, glycerin, mineral oil, oleic acid, fatty acid esters, benzyl alcohol, an alcohol, and any combination thereof, preferably coconut oil, hydrogenated vegetable oils, ethanol, and any combination thereof.

In certain embodiments, the solvent is present in an amount of about 0% to about 3% by weight.

Exemplary pharmaceutically acceptable adsorbents include silica, microcrystalline cellulose, cellulose, silicified microcrystalline cellulose, starch, pregelatinized starch, dicalcium phosphate, and any combinations thereof.

The formulations disclosed herein may further comprise one or more fillers or excipients (e.g., lactose, sugar, mannitol, sorbitol, xylitol, or any other sugar alcohol). In certain embodiments, the excipient is a filler.

Fillers or excipients may be present in an amount of about 0% to about 90% by weight or about 1% to about 80% by weight.

The excipient may be a disintegrant (e.g., starch, sodium starch glycolate, pregelatinized starch, cross-linked polyvinyl pyrrolidone, cross linked calcium or sodium carboxy methyl cellulose, low-substituted hydroxypropyl cellulose, microcrystalline cellulose, ion exchange resin, cross-linked polyacrylic acid, alginates, colloidal magnesium-aluminum silicate, or calcium silicate, preferably cross-linked polyvinyl pyrrolidone or calcium silicate).

In certain embodiments, the disintegrant is present in an amount of about 0.25% to about 50% by weight, about 0.5% to about 30% by weight, or about 1% to about 20% by weight.

In certain embodiments, the formulation disclosed herein further comprises one or more other pharmaceutically acceptable excipients (e.g., diluents, lubricants, granulating aids, colorants, flavorants, flavors, surfactants, pH adjusters, anti-adherents, and glidants).

Exemplary lubricants include magnesium stearate, stearic acid, palmitic acid, calcium stearate, talc, polyethylene glycol, colloidal silicon dioxide, silicon dioxide, sodium stearyl fumarate, carnauba wax, and mixtures thereof.

In certain embodiments, the lubricants are present in an amount of about 0.2% to about 8% by weight or about 0.5% to about 2.5% by weight.

Exemplary flavorants or flavors include vanilla, strawberry, cherry, grape, lemon, lime, orange, peppermint, spearmint, cinnamon, and mixtures thereof.

In certain embodiments, the flavorants or flavors are present in an amount of about 0.005% to about 20% by weight or about 0.01% to about 5% by weight.

The cannabinoid may be an extract from a cannabis plant.

In certain embodiments, the formulation has a combination of at least two cannabinoids. In certain embodiments, the formulation comprises at least two cannabinoids.

The two cannabinoids or the at least two cannabinoids may be selected from Tetrahydrocannabinolic acid (THCa), Cannabidiolic acid (CBDa), Cannabinolic acid (CBNa), Cannabichromenic acid (CBCa), Tetrahydrocannabinol (THC), Cannabidiol (CBD), Cannabigerol (CBG), Cannabigerolic Acid (CBGa), Cannabichromene (CBC), Cannabinol (CBN), Cannabielsoin (CBE), iso-Tetrahydrocannabinol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM), and derivatives thereof.

The two cannabinoids may be in a 1:1 proportion by weight, in a 10:1 proportion by weight, or in a 20:1 proportion by weight.

The two cannabinoids may be THC and CBD, or the two cannabinoids may be THCa and CBDa.

The at least one cannabinoid may be THC or THCa present in an amount ranging from about 0.01 mg to about 200 mg, or the at least one cannabinoid may be CBD or CBDa present in an amount ranging from about 0.01 mg to about 200 mg.

The total cannabinoid content may form about 0.05% to about 5%, e.g., about 1.1%, by weight based on 100 parts by weight of the formulation.

In another aspect, provided herein are formulations comprising:

a. CBD and THC cannabinoid extract,

b. coconut oil,

c. microcrystalline cellulose,

d. mannitol,

e. sodium starch glycolate,

f. silicon dioxide,

g. magnesium stearate, and

h. a flavor.

Components of the formulation may exist in the following percentage:

a. the CBD and THC cannabinoid extract is present in amount of about 3.8% by weight;

b. the coconut oil is present in amount of about 1.9% by weight;

c. the microcrystalline cellulose is present in amount of about 20.0% by weight;

d. the mannitol is present in amount of about 65.8% by weight;

e. the sodium starch glycolate is present in amount of about 5.0% by weight;

f. the silicon dioxide is present in amount of about 2.0% by weight;

g. the magnesium stearate is present in amount of about 0.5% by weight; and

h. the flavor is present in amount of about 1.0% by weight.

In another aspect, provided herein are formulations comprising:

a. a cannabinoid extract comprising CBD and THC,

b. coconut oil,

c. microcrystalline cellulose,

d. mannitol,

e. sodium starch glycolate,

f. silicon dioxide,

g. magnesium stearate, and

h. a flavor.

Components of the formulation may exist in the following percentage:

a. the cannabinoid extract comprising CBD and THC is present in amount of about 3.8% by weight;

b. the coconut oil is present in amount of about 1.9% by weight;

c. the microcrystalline cellulose is present in amount of about 20.0% by weight;

d. the mannitol is present in amount of about 65.8% by weight;

e. the sodium starch glycolate is present in amount of about 5.0% by weight;

f. the silicon dioxide is present in amount of about 2.0% by weight;

g. the magnesium stearate is present in amount of about 0.5% by weight; and

h. the flavor is present in amount of about 1.0% by weight.

The formulation may be in the form of a tablet, a capsule, a pellet, a granule, a powder, a coated granule, or a coated pellet.

In a further aspect, provided are methods for treating a disease or a disorder, the method comprising administering (e.g., administering orally) to a subject a formulation disclosed herein.

The disease or disorder may be selected from a skin disease or disorder, pain associated with cancer, neuropathic pain and HIV-associated sensory neuropathy, side effects of chemotherapy including nausea and pain, symptoms of neurological and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis, and dystonia.

In yet another aspect, provided herein are methods of manufacturing of a formulation disclosed herein, the method comprising the steps of:

providing one or more cannabinoids;

providing one or more solvents;

providing one or more pharmaceutically acceptable adsorbents;

providing one or more fillers, excipients, disintegrants, or combinations thereof; and mixing the one or more cannabinoids or derivatives thereof, the one or more solvents, the one or more pharmaceutically acceptable adsorbents, and the one or more fillers, excipients, disintegrants, or combinations thereof.

Other features and advantages of the present invention will become apparent from the following detailed description, examples, and figures. It should be understood, however, that the detailed description and the specific examples, while indicating embodiments of the invention, are given by way of illustration only, since various changes and modifications within the spirit and scope of the invention will become apparent to those skilled in the art from this detailed description.

### DETAILED DESCRIPTION OF THE INVENTION

The present subject matter may be understood more readily by reference to the following detailed description which forms a part of this disclosure. It is to be understood that this invention is not limited to the specific products, methods, conditions, or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality,” as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “treatment” or “therapy” (as well as different forms thereof) include preventative (e.g., prophylactic), curative, or palliative treatment. As used herein, the term “treating” includes alleviating or reducing at least one adverse or negative effect or symptom of a condition, disease, or disorder.

The terms “subject,” “individual,” and “patient” are used interchangeably herein, and refer to an animal, for example a human, to whom treatment, including prophylactic treatment, with the pharmaceutical formulation according to the present invention, is provided.

The inventor of the instant application surprisingly and unexpectedly discovered a formulation which includes, in part, one or more cannabinoids in combination with one or more solvents or pharmaceutically acceptable adsorbents.

In certain embodiments, the present application provides a tablet with optimal mechanical strength, which when placed in the oral cavity rapidly dissolves or disintegrates without water.

In certain embodiments, the formulation is an orally disintegrating or dissolving tablet composition of a cannabinoid ingredient, which rapidly disintegrates or dissolves in the oral mucosa and ensures uniformity of dose and desired therapeutic outcome.

The tablet formulations of the present application exhibit low friability, low ejection forces, and hardness sufficient to be processed in high speed tableting machines, while retaining rapid disintegration or dissolution properties. The tablet formulations have a pleasant mouth feel and good mechanical strength such that they do not require special handling or packaging conditions.

Certain embodiments relate to compositions of one or more cannabinoids to provide, for example, an oral delivery of said one or more cannabinoids. The cannabinoids are easily prepared and formulated to provide consistent, therapeutically effective dosage forms from lot to lot.

In some embodiments, the cannabinoid is a cannabinoid extract that contains a combination of at least two of the following: Tetrahydrocannabinolic acid (THCa), Cannabidiolic acid (CBDa), Cannabinolic acid (CBNa), Cannabichromenic acid (CBCa), Tetrahydrocannabinol (THC), Cannabidiol (CBD), Cannabigerol (CBG), Cannabigerolic Acid (CBGa), Cannabichromene (CBC), Cannabinol (CBN), Cannabielsoin (CBE), iso-Tetrahydrocannabinol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM), and derivatives thereof. The cannabinoid may be natural or synthetic.

The methods of making cannabinoids extract is well known in the art. The cannabis plants are grown, harvested, and the cannabinoids are extracted through, for example, a CO2 extraction process.

In some embodiments, the formulation includes a first cannabinoid and a second cannabinoid. In certain examplary embodiments, the first and second cannabinoids are in a ratio ranging from about 1:1 to about 20:1 proportion by weight. In other embodiments, the first and second cannabinoids are in a 1:1 proportion by weight. In other embodiments, the first and second cannabinoids are in a 10:1 proportion by weight. In yet other embodiments, the first and second cannabinoids are in a 20:1 proportion by weight.

In certain embodiments, the first cannabinoid is THC or THCa and the second cannabinoid is CBD or CBDa.

The first cannabinoid can be present in an amount ranging from about 0.01 mg to about 200 mg. The second cannabinoid also can be present in an amount ranging from about 0.01 mg to about 200 mg.

In some embodiments, the cannabinoids are in equal proportion (e.g., equal proportion by weight) such as, for example, 2.5 mg THC to 2.5 mg CBD. Other embodiments include proportions of THC/CBD of 0.25 mg THC to 5.0 mg CBD or 5.0 mg THC to 0.25 mg CBD.

In some embodiments, the cannabinoid is any member of a group of substances that are structurally related to tetrahydrocannabinol and that bind to a cannabinoid receptor such as CB1 or CB2 or both (‘THC’). The cannabinoid can be a naturally occurring compound (e.g., present in cannabis), a compound metabolized by a plant or animal, or a synthetic derivative.

The cannabinoid may be included in its free form or in the form of a salt; an acid addition salt of an ester; an amide; an enantiomer; an isomer; a tautomer; a prodrug; a derivative of an active agent; different isomeric forms (e.g., enantiomers and diastereoisomers), both in pure form and in admixture, including racemic mixtures; or enol forms.

Cannabinoids suitable for use with the present disclosure encompass natural cannabinoids, natural cannabinoids that have been purified or modified, and synthetically derived cannabinoids. Methods for purifying cannabinoids, obtained from plant material, are well known in the art and are fully described in, for example, United States Patent Application Publication No. 2005/0266108, which is incorporated by reference herein in its entirety.

The cannabinoids can be any of 9-tetrahydrocannabinol, 8-tetrahydrocannabinol, (+)-1,1-dimethylheptyl analog of 7-hydroxy-delta-6-tetrahydrocannabinol, 3-(5′-cyano-1′,1′-dimethylpentyl)-1-(4-N-morpholinobutyryloxy)-delta-8-tetrahydrocannabinol hydrochloride, dexanabinol, nabilone, levonantradol, and N-(2-hydroxyethyl)hexadecanoamide. In some embodiments, the cannabinoids can be any of the non-psychotropic cannabinoid 3-dimethylnepty 11 carboxylic acid homologine 8 and delta-8-tetrahydrocannabinol.

In another aspect, provided are formulations comprising at least one cannabinoid or a derivative thereof, a solvent, a pharmaceutically acceptable adsorbent, one or more fillers or excipients, a disintegrant, or combinations thereof. In certain embodiments, the formulation further comprise diluents, lubricants, granulating aids, colorants, flavorants, flavors, surfactants, pH adjusters, anti-adherents, glidants, or combinations thereof.

In certain embodiments, the formulation comprises: at least one cannabinoid or a derivative thereof, a solvent, a pharmaceutically acceptable adsorbent, one or more fillers or excipients, a disintegrant, diluents, lubricants, granulating aids, colorants, flavorants, flavors, surfactants, pH adjusters, anti-adherents, or glidants, or combinations thereof.

In some embodiments, the minor and major phyto-compounds in the formulation are used in an amount to give a content of from 0.05% to 5% by weight based on 100 parts by weight of the formulation. In other embodiments, the minor and major phyto-compounds in the formulation are used in an amount to give a content of about 1.1% by weight based on 100 parts by weight of the formulation.

In some embodiments, the total cannabinoid content used in the formulation gives a content of from 0.05% to 5% by weight based on 100 parts by weight of the formulation. In other embodiments, the total cannabinoid content used in the formulation gives a content of about 1.1% by weight based on 100 parts by weight of the formulation.

The formulation may also include additional ingredients such as solvents, carriers, or excipients.

Examples of the solvents include, but are not limited to, vegetable oil, hydrogenated vegetable oils, coconut oil, limonene, a terpene, an essential oil, polyethylene glycols (PEG), propylene glycol, ethanol, substituted polyethylene glycols, glycerin, mineral oil, oleic acid, fatty acid esters, benzyl alcohol, an alcohol, or combinations thereof

In certain embodiments, the total solvent content in the formulation is used in an amount to give a content of from 0% to 5% by weight based on 100 parts by weight of the formulation. In other embodiments, the total solvent content in the formulation is used in an amount to give a content of about 1.9% by weight based on 100 parts by weight of the formulation. In other embodiments, the total solvent content in the formulation is used in an amount to give a content of about 1% to 20% by weight based on 100 parts by weight of the formulation. In other embodiments, the total solvent content in the formulation is used in an amount to give a content of about 1% to 40% by weight based on 100 parts by weight of the formulation.

Examples of a terpene include, but are not limited to, beta-myrcene, limonene, beta caryopyllene, caryopyllene oxide, terpineol, citronellol, linalool, humulene, beta-amyrin, cycloartenol, farnesol, menthol, eucalyptol, eugenol, and borneol.

Examples of pharmaceutically acceptable adsorbents include, for example, but are not limited to, silica, microcrystalline cellulose, cellulose, silicified microcrystalline cellulose, starch, pregelatinized starch, dicalcium phosphate, or mixtures thereof. In certain embodiments, the total adsorbents content in the formulation is used in an amount to give a content of about 1% to about 40.0% by weight based on 100 parts by weight of the formulation. In other embodiments, the total adsorbents content in the formulation is used in an amount to give a content of about 20.0% to about 30.0% by weight based on 100 parts by weight of the formulation. In other embodiments, the total adsorbents content in the formulation is used in an amount to give a content of about 20.0% by weight based on 100 parts by weight of the formulation.

Examples of fillers or excipients include, but are not limited to, lactose, sugar, mannitol, sorbitol, xylitol, or any other sugar alcohol. The used amount of the fillers or excipients is an amount to give a content of from about 0% to about 90% by weight or from about 1% to about 80% by weight. In certain embodiments, the total fillers content in the formulation is used in an amount to give a content of about 65.8% by weight based on 100 parts by weight of the formulation.

In one embodiments, the excipient used in the formulation is a disintegrant.

Examples of disintegrants include, but are not limited to, starch, sodium starch glycolate, pregelatinized starch, crosslinked polyvinyl pyrrolidone, cross linked calcium or sodium carboxy methyl cellulose, low-substituted hydroxypropyl cellulose, microcrystalline cellulose, ion exchange resin, cross-linked polyacrylic acid, alginates, colloidal magnesium-aluminum silicate, or calcium silicate. The used amount of the disintegrants is an amount to give a content of from about 0.25% to about 50% by weight or from about 0.5% to about 30% by weight or from about 1% to about 20% by weight. In certain embodiments, the total disintegrants content in the formulation is used in an amount to give a content of about 5.0% by weight based on 100 parts by weight of the formulation.

Examples of other pharmaceutically acceptable excipients include, but are not limited to, diluents, lubricants, granulating aids, colorants, flavorants, flavors, surfactants, pH adjusters, anti-adherents, or glidants.

Examples of colorants include, but are not limited to, a food dye (e.g., food yellow No. 5, food red No. 2, food blue No. 2), a food lake color, iron oxide red, and iron oxide yellow.

Examples of the pH adjusters include, but are not limited to, citrate, phosphate, carbonate, tartrate, fumarate, acetate, and amino acid salt.

Examples of lubricants include, but are not limited to, magnesium stearate, stearic acid, palmitic acid, calcium stearate, talc, polyethylene glycol, colloidal silicon dioxide, silicon dioxide, sodium stearyl fumarate, carnauba wax, or mixtures thereof. The used amount of the lubricants is an amount to give a content of from about 0.2% to about 8% by weight or from about 0.5% to about 2.5% by weight. In certain embodiments, the total lubricants content in the formulation is used in an amount to give a content of about 2.5% by weight based on 100 parts by weight of the formulation.

Examples of flavorants or flavors include, but are not limited to, vanilla, strawberry, cherry, grape, lemon, lime, orange, peppermint, spearmint, cinnamon, or mixtures thereof. The used amount of the flavorants or flavors is an amount to give a content of from about 0.005% to about 20% by weight or from about 0.01% to about 5% by weight.

The above ingredients may be used by combining two or more members at an appropriate ratio.

The formulations disclosed herein include a composition for daily administration. In certain embodiments, the therapeutic effect is maintained with one unit, twice daily. In other embodiments, the therapeutic effect is maintained with one unit, three times daily. In certain exemplary embodiments, the duration of each dose's effect is between four (4) to six (6) hours.

The formulation can be of any suitable form, for example, tablets, capsules, pellets, granules, powders, coated granules, or coated pellets.

Examples of a disease or a disorder that can be treated by the invention include, but are not limited to, a skin disease or disorder, pain associated with cancer, neuropathic pain and HIV-associated sensory neuropathy, side effects of chemotherapy including nausea and pain, symptoms of neurological and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis, and dystonia.

### Manufacturing

In yet another aspect, provided herein are methods for the preparation of a formulation of tablets containing a combination of cannabinoids.

The tablets of the present invention may be manufactured using any method known in the art. In certain embodiments, the method of manufacturing comprises an indirect tableting method wherein active ingredients and various additives are granulated through either fluidized bed granulation, agitation granulation, rolling granulation, rolling fluidized granulation, or extruding granulation, and mixed with the lubricant as desired to obtain a powder for tableting. In certain embodiments, the method of manufacture of the tablets of the present invention comprises the steps of providing at least one cannabinoid extract from a cannabis plant; combining the extract with a pharmaceutically acceptable carrier in a solvent to form granules using a high shear granulation means; drying the granules; blending the dried granules with sodium starch glycolate to form a powder blend; and forming the tablets. In other embodiments, the cannabinoid extract is combined with microcrystalline cellulose and ethanol in a high shear granulation process. The resulting granules are then allowed to dry to remove the solvent prior to forming the tablets.

The tablets of the present invention can be formed using any method known in the art. In certain embodiments, the tablets are formed by hard pressing. In other embodiments, the tablets are formed with a tableting machine, such as a rotary tableting machine or a high-productivity single shot tableting machine. In certain embodiments, a well-known direct tableting method is used, wherein a mixture of active ingredients and various additives acts as a powder for tableting, and is tableted with a tableting machine, for example a rotary tableting machine or a high-productivity single shot tableting machine. When compression molding is used, lubricants may be applied prior to the punch and die step of tableting machine, and then compression molding may be performed without mixing the lubricant with the granules in the indirect tableting method or the mixture in the direct tableting method.

All patents and literature references cited in the present specification are hereby incorporated by reference in their entirety.

The present invention will be specifically explained by way of examples, but these examples are not intended to limit the present invention.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. Tablet Formulation Having Cannabinoid

Formulations described above are shown in Table 1 and are prepared using the methods described herein.

The formulation is an orally disintegrating or dissolving tablet composition of a cannabinoid ingredient, which rapidly disintegrates or dissolves in the oral mucosa and ensures uniformity of dose and desired therapeutic outcome.

The formulation includes a mixture of a therapeutic agent (e.g., cannabinoid extract), solvents, pharmaceutically acceptable adsorbents, and fillers or excipients.

The formulation is suitable for oral administration.

TABLE 1
The formulation having CBD and THC
cannabinoid extract

| | Concentration |
|---|---|
| Ingredients | (% w/w) |
| CBD and THC cannabinoid extract | 3.8 |
| Coconut oil | 1.9 |
| Microcrystalline cellulose | 20.0 |
| Mannitol | 65.8 |
| Sodium starch glycolate | 5.0 |
| Silicon dioxide | 2.0 |
| Magnesium stearate | 0.5 |
| Flavor | 1.0 |
| Total | 100.0 |

Unless defined otherwise, all technical and scientific terms used herein have the same meanings as commonly understood by one of ordinary skill in the art.

Having described embodiments of the present invention with reference to the accompanying drawings, it is to be understood that the present invention is not limited to the above-mentioned embodiments and that various changes and modifications can be affected by one skilled in the art without departing from the spirit or scope of the present invention as defined in the appended claims.

We claim:

1. A formulation comprising:
a. at least one cannabinoid or a derivative thereof,
b. a solvent, and
c. a pharmaceutically acceptable adsorbent.

2. The formulation of claim 1, wherein the solvent is a vegetable oil, hydrogenated vegetable oils, coconut oil, limonene, a terpene, an essential oil, polyethylene glycols (PEG), propylene glycol, ethanol, substituted polyethylene glycols, glycerin, mineral oil, oleic acid, fatty acid esters, benzyl alcohol, an alcohol, or any combination thereof.

3. The formulation of claim 1, where the solvent is coconut oil, hydrogenated vegetable oil, ethanol, or any combination thereof.

4. The formulation of any one of claims 1-3, wherein the solvent is present in an amount of about 0% to about 3% by weight.

5. The formulation of any one of claims 1-4, wherein the pharmaceutically acceptable adsorbent is silica, microcrystalline cellulose, cellulose, silicified microcrystalline cellulose, starch, pregelatinized starch, dicalcium phosphate, or any combination thereof.

6. The formulation of any one of claims 1-5, further comprising one or more fillers or excipients.

7. The formulation of claim 6, wherein the fillers or excipients are lactose, sugar, mannitol, sorbitol, xylitol, or any other sugar alcohol.

8. The formulation of claim 6 or 7, wherein the fillers or excipients are present in an amount of about 0% to about 90% by weight.

9. The formulation of claim 6 or 7, wherein the fillers or excipients are present in an amount of about 1% to about 80% by weight.

10. The formulation of claim 6, wherein the excipient is a disintegrant.

11. The formulation of claim 10, wherein the disintegrant is starch, sodium starch glycolate, pregelatinized starch, cross-linked polyvinyl pyrrolidone, cross linked calcium or sodium carboxy methyl cellulose, low-substituted hydroxypropyl cellulose, microcrystalline cellulose, ion exchange resin, cross-linked polyacrylic acid, alginates, colloidal magnesium-aluminum silicate, or calcium silicate.

12. The formulation of claim 11, wherein the disintegrant is cross-linked polyvinyl pyrrolidone or calcium silicate.

13. The formulation of any one of claims 10-12, wherein the disintegrant is present in an amount of about 0.25% to about 50% by weight.

14. The formulation of any one of claims 10-12, wherein the disintegrant is present in an amount of about 0.5% to about 30% by weight.

15. The formulation of any one of claims 10-12, wherein the disintegrant is present in an amount of about 1% to about 20% by weight.

16. The formulation of any one of claims 1-15, further comprising one or more other pharmaceutically acceptable excipients.

17. The formulation of claim 16, wherein the one or more other pharmaceutically acceptable excipients are selected from diluents, lubricants, granulating aids, colorants, flavorants, flavors, surfactants, pH adjusters, anti-adherents, and glidants.

18. The formulation of claim 17, wherein the lubricants are selected from magnesium stearate, stearic acid, palmitic acid, calcium stearate, talc, polyethylene glycol, colloidal silicon dioxide, silicon dioxide, sodium stearyl fumarate, carnauba wax, and mixtures thereof.

19. The formulation of claim 18, wherein the lubricants are present in an amount of about 0.2% to about 8% by weight.

20. The formulation of claim 18, wherein the lubricants are present in an amount of about 0.5% to about 2.5% by weight.

21. The formulation of claim 17, wherein the flavorants or flavors are selected from vanilla, strawberry, cherry, grape, lemon, lime, orange, peppermint, spearmint, cinnamon, and mixtures thereof.

22. The formulation of claim 21, wherein the flavorants or flavors are present in an amount of about 0.005% to about 20% by weight.

23. The formulation of claim 21, wherein the flavorants or flavors are present in an amount of about 0.01% to about 5% by weight.

24. The formulation of any one of claims 1-23, wherein the cannabinoid is an extract from a cannabis plant.

25. The formulation of any one of claims 1-24, wherein the formulation has a combination of at least two cannabinoids.

26. The formulation of claim 25, wherein the two cannabinoids are selected from Tetrahydrocannabinolic acid (THCa), Cannabidiolic acid (CBDa), Cannabinolic acid (CBNa), Cannabichromenic acid (CBCa), Tetrahydrocannabinol (THC), Cannabidiol (CBD), Cannabigerol (CBG), Cannabigerolic Acid (CBGa), Cannabichromene (CBC), Cannabinol (CBN), Cannabielsoin (CBE), iso-Tetrahydrocannabinol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM), and derivatives thereof.

27. The formulation of claim 25 or 26, wherein the two cannabinoids are in a 1:1 proportion by weight.

28. The formulation of claim 25 or 26, wherein the two cannabinoids are in a 10:1 proportion by weight.

29. The formulation of claim 25 or 26, wherein the two cannabinoids are in a 20:1 proportion by weight.

30. The formulation of any one of claims 25-29, wherein the two cannabinoids are THC and CBD.

31. The formulation of any one of claims 25-29, wherein the two cannabinoids are THCa and CBDa.

32. The formulation of any one of claims 1-31, wherein the at least one cannabinoid is THC or THCa present in an amount ranging from about 0.01 mg to about 200 mg.

33. The formulation of any one of claims 1-31, wherein the at least one cannabinoid is CBD or CBDa present in an amount ranging from about 0.01 mg to about 200 mg.

34. The formulation of any one of claims 1-33, wherein a total cannabinoid content forms about 0.05% to 5% by weight based on 100 parts by weight of the formulation.

35. The formulation of any one of claims 1-33, wherein a total cannabinoid content forms about 1.1% by weight based on 100 parts by weight of the formulation.

36. A formulation comprising:
a. CBD and THC cannabinoid extract,
b. coconut oil,
c. microcrystalline cellulose,
d. mannitol,
e. sodium starch glycolate,
f. silicon dioxide,
g. magnesium stearate, and
h. a flavor.

37. The formulation of claim 36, wherein:
a. the CBD and THC cannabinoid extract is present in amount of about 3.8% by weight;
b. the coconut oil is present in amount of about 1.9% by weight;
c. the microcrystalline cellulose is present in amount of about 20.0% by weight;
d. the mannitol is present in amount of about 65.8% by weight;
e. the sodium starch glycolate is present in amount of about 5.0% by weight;
f. the silicon dioxide is present in amount of about 2.0% by weight;
g. the magnesium stearate is present in amount of about 0.5% by weight; and
h. the flavor is present in amount of about 1.0% by weight.

38. The formulation of claim 36 or 37, wherein the formulation is in the form of tablet, capsule, pellet, granule, powder, coated granule, or coated pellet.

39. A method for treating a disease or a disorder, the method comprising administering to a subject a formulation of any one of claims 1-38.

40. The method of claim 39, wherein the disease or the disorder is selected from a skin disease or disorder, pain associated with cancer, neuropathic pain and HIV-associated sensory neuropathy, side effects of chemotherapy including nausea and pain, symptoms of neurological and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis, and dystonia.

41. The method of claim 39 or 40, wherein the formulation is administered orally.

42. A method of manufacturing of a formulation of any of claims 1-38, the method comprising the steps of
providing one or more cannabinoids;
providing one or more solvents;
providing one or more pharmaceutically acceptable adsorbents;
providing one or more fillers, excipients, disintegrants, or combinations thereof; and
mixing the one or more cannabinoids or derivatives thereof, the one or more solvents, the one or more pharmaceutically acceptable adsorbents, and the one or more fillers, excipients, disintegrants, or combinations thereof.
